(19) United States
(12) Patent Application Publication
(10) Pub. No.: US 2024/0294975 A1
(43) Pub. Date: Sep. 5, 2024

(54) SPATIAL ANALYSIS UTILIZING DEGRADABLE HYDROGELS

(71) Applicant: 10x Genomics, Inc., Pleasanton, CA (US)

(72) Inventors: Bill Kengli Lin, Pleasanton, CA (US); Jill Herschleb, San Francisco, CA (US); Cedric Uytingco, Milpitas, CA (US)

(21) Appl. No.: 18/662,508

(22) Filed: May 13, 2024

Related U.S. Application Data
(62) Division of application No. 17/368,259, filed on Jul. 6, 2021, now Pat. No. 11,981,960.
(60) Provisional application No. 63/048,413, filed on Jul. 6, 2020.

(51) Int. Cl. C12Q 1/6841; C12Q 1/6837

(52) U.S. Cl. CPC C12Q 1/6841; C12Q 1/6837

(57) ABSTRACT

Provided are methods of capturing an analyte from a biological sample using a hydrogel that includes capture probes to capture the analyte, identifying a region of interest of the biological sample, and isolating the region of interest from the biological sample by removing a portion of the hydrogel that corresponds to the region of interest. Compositions and kits for performing the methods are also provided.

### CROSS-REFERENCE TO RELATED APPLICATIONS

This application is a divisional application from patent application Ser. No. 17/368,259, filed Jul. 6, 2021, which claims priority to U.S. Provisional Patent Application No. 63/048,413, filed Jul. 6, 2020. The entire content of the foregoing application is incorporated herein by reference.

### BACKGROUND

Cells within a tissue have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling, and cross-talk with other cells in the tissue.

Spatial heterogeneity has been previously studied using techniques that typically provide data for a handful of analytes in the context of intact tissue or a portion of a tissue (e.g., tissue section), or provide significant analyte data from individual, single cells, but fails to provide information regarding the position of the single cells from the originating biological sample (e.g., tissue).

Selecting a region of interest to process rather than processing the entire biological sample eliminates extraneous downstream processing (e.g., sequencing) of regions that are not of interest, which provides cost, resource and time savings. Thus, there is a need to develop methods for selecting a region of interest for spatial transcriptomics.

### SUMMARY

Provided herein are methods and compositions for selecting a region of interest in a biological sample prior to or during spatial transcriptomics. The methods and compositions herein utilize a hydrogel that is modifiable e.g., by degradation, by chemical means, or by physical means in order to expose one or more regions of interest for analyte detection using spatial transcriptomics. The methods and compositions utilized herein provide the advantage of reducing downstream reagents, downstream experimental time and settings, and increasing targeted analysis (e.g., targeted analyte analysis) at one or more regions of interest in a biological sample. The methods and compositions allow a user to select the size (e.g., area) of a biological sample that is the region of interest (or that is not the region of interest), and this size can vary based on tissue size, makeup, and pathology.

Accordingly, provided herein are methods of determining a location and/or an abundance of an analyte at a region of interest from a biological sample, the method comprising: (a) aligning the biological sample with a substrate comprising a hydrogel layered over a plurality of capture probes, wherein a capture probe of the plurality of capture probes comprises a spatial barcode and a capture domain; (b) identifying the region of interest in the biological sample; (c) degrading a portion of the hydrogel corresponding to the region of interest of the biological sample, thereby exposing the capture probe; (d) contacting the biological sample with the substrate; (e) hybridizing the analyte at the region of interest to the capture domain; and (f) determining (i) all or a portion of a sequence of the spatial barcode, or a complement thereof, and (ii) all or a portion of a sequence of the analyte, or a complement thereof, and using the sequences of (i) and (ii) to determine the location and/or the abundance of the analyte in the region of interest in the biological sample.

In some embodiments, degrading the portion of the hydrogel comprises exposing the region of interest to UV light, laser light, natural light, a heating device, a radiation device, a plasma device, a microwave device or a degradation agent, wherein the degradation agent degrades the portion of the hydrogel underneath the biological sample.

In some embodiments, the method described herein further comprises dissolving the hydrogel at regions outside of the region of interest.

In some embodiments, the method described herein further comprises, at the regions outside of the region of interest: hybridizing an analyte outside of the region of interest to a capture domain outside of the region of interest; and determining (i) all or a portion of a sequence of a spatial barcode of the capture domain of the region of interest, or a complement thereof, and (ii) all or a portion of a sequence of the analyte outside of the region of interest, or a complement thereof, and using the sequences of (i) and (ii) to determine the location and/or the abundance of the analyte outside of the region of interest in the biological sample.

In some embodiments, the hydrogel is layered over the substrate prior to contacting the biological sample with a substrate.

In some embodiments, degrading the portion of the hydrogel corresponding to the region of interest comprises treating the hydrogel at the region of interest with a reducing agent.

In some embodiments, the reducing agent comprises dithiothreitol.

In some embodiments, the treating the hydrogel at the region of interest with a reducing agent is performed before step (d)

In some embodiments, step (d) is performed before steps (b) and (c).

In some embodiments, the treating the hydrogel at the region of interest with a reducing agent is performed after step (d).

In some embodiments, the biological sample is stained using hematoxylin and eosin (H&E), immunofluorescence, or immunohistochemistry.

In some embodiments, the hydrogel comprises hydrogel subunits selected from the group consisting of: acrylamide, bis-acrylamide, polyacrylamide and derivatives thereof, poly(ethylene glycol) and derivatives thereof, gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, or any combination thereof.

In some embodiments, the hydrogel comprises one or more permeabilization reagent, wherein the permeabilization reagent comprises pepsin or proteinase K.

In some embodiments, the capture probe further comprises one or more functional domains, a unique molecular identifier, a cleavage domain, or any combination thereof, wherein the cleavage domain is a cleavable linker selected from the group consisting of a photocleavable linker, a UV-cleavable linker, an enzymatic cleavable linker, and a pH-sensitive cleavable linker.

In some embodiments, the biological sample is a fresh frozen tissue sample or formalin fixed paraffin embedded (FFPE) tissue sample.

In some embodiments, the analyte is an RNA molecule.

In another aspect, provided herein are kits comprising: (a) a substrate comprising a hydrogel, wherein the substrate comprises a plurality of capture probes, wherein a capture probe of the plurality of capture probes comprises a spatial barcode and a capture domain; (b) one or more enzymes selected from a reverse transcriptase, a DNA polymerase, or both; (c) one or more reagents for reverse transcription or second strand synthesis (d) a reducing agent; and (e) instructions for performing any one of the methods described herein.

In another aspect, provided herein are compositions comprising: (a) a substrate comprising a hydrogel, wherein the substrate comprises a plurality of capture probes, wherein a capture probe of the plurality of capture probes comprises a spatial barcode and a capture domain; (b) one or more enzymes selected from a reverse transcriptase, a DNA polymerase, or both; (c) one or more reagents for reverse transcription or second strand synthesis; (d) a reducing agent; and (e) a biological sample.

In another aspect, provided herein are methods of determining a location and/or an abundance of an analyte at a region of interest from a biological sample, the method comprising: (a) contacting the biological sample with a substrate comprising a hydrogel, wherein the substrate comprises a plurality of capture probes, wherein a capture probe of the plurality of capture probes comprises a spatial barcode and a capture domain; (b) hybridizing a plurality of analytes to the plurality of capture domains, wherein the plurality of analytes comprises the analyte at the region of interest; (c) identifying the region of interest in the biological sample; (d) isolating the region of interest of the biological sample; and (e) determining (i) all or a portion of a sequence of the spatial barcode, or a complement thereof, and (ii) all or a portion of a sequence of the analyte, or a complement thereof, and using the sequences of (i) and (ii) to determine the location and/or the abundance of the analyte in the region of interest in the biological sample.

In some embodiments, isolating the region of interest of the biological sample comprises removing a portion of the hydrogel that corresponds to the region of interest using a mechanical hole punch, microsurgery, laser capture microdissection, mechanically cutting the region of interest from the biological sample, or any combination thereof

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

The singular form “a”, “an”, and “the” include plural references unless the context clearly dictates otherwise. For example, the term “a cell” includes one or more cells, comprising mixtures thereof. “A and/or B” is used herein to include all of the following alternatives: “A”, “B”, “A or B”, and “A and B”.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 is a schematic diagram showing an example of a barcoded capture probe, as described herein.

FIG. 2 is a schematic illustrating a cleavable capture probe, wherein the cleaved capture probe can enter into a non-permeabilized cell and bind to target analytes within the sample.

FIG. 3 is a schematic diagram of an exemplary multiplexed spatially-barcoded feature.

FIG. 4 is a schematic showing the arrangement of barcoded features within an array.

FIG. 5 is a schematic illustrating a side view of a diffusion-resistant medium.

FIGS. 6A and 6B are schematics illustrating expanded FIG. 6A and side views FIG. 6B of an electrophoretic transfer system configured to direct transcript analytes toward a spatially-barcoded capture probe array.

FIGS. 7A-7G show a schematic illustrating an exemplary workflow protocol utilizing an electrophoretic transfer system.

FIG. 8 shows a schematic of an example analytical workflow in which electrophoretic migration of analytes is performed in conjunction with a permeabilization step.

FIG. 9A shows an example perpendicular, single slide configuration for use during electrophoresis.

FIG. 9B shows an example parallel, single slide configuration for use during electrophoresis.

FIG. 9C shows an example multi-slide configuration for use during electrophoresis.

FIG. 10 shows an example workflow for laser capture microdissection.

FIG. 11 shows an example workflow using a hydrogel onto which is deposited a plurality of capture probes.

FIG. 12 shows an example workflow using a substrate comprising a plurality of capture probes over which is layered a hydrogel and isolating a region of interest for analyte capture from a biological sample using one method for gel degradation.

FIG. 13 shows an example workflow using a substrate comprising a plurality of capture probes over which is layered a hydrogel and degrading of a portion of the hydrogel corresponding to a region of interest in a biological sample by using a second method for gel degradation.

### DETAILED DESCRIPTION

### I. Methods and Compositions for Spatial Analysis

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodriques et al., Science 363(6434):1463-1467, 2019; Lee et al., Nat. Protoc. 10(3):442-458, 2015; Trejo et al., PLoS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination. Further non-limiting aspects of spatial analysis methodologies and compositions are described herein.

Some general terminology that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)). See, e.g., Section (II)(b)(e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

In some embodiments, the capture probe further includes a unique molecular identifier. In some embodiments, the capture probe further includes a functional domain. In some embodiments, the capture domain includes a sequence that is partially complementary to the analyte or an analyte binding moiety. In some embodiments, the capture domain includes a homopolymeric sequence. In some embodiments, the capture domain includes a poly(T) sequence. In some embodiments, the capture probe further includes a cleavage domain. In some embodiments, the cleavage domain includes a cleavable linker. Non-limiting examples of a cleavable linker include a photocleavable linker, a UV-cleavable linker, an enzymatic cleavable linker, or a pH-sensitive cleavable linker. In some embodiments, the plurality of capture probes are attached to one or more features.

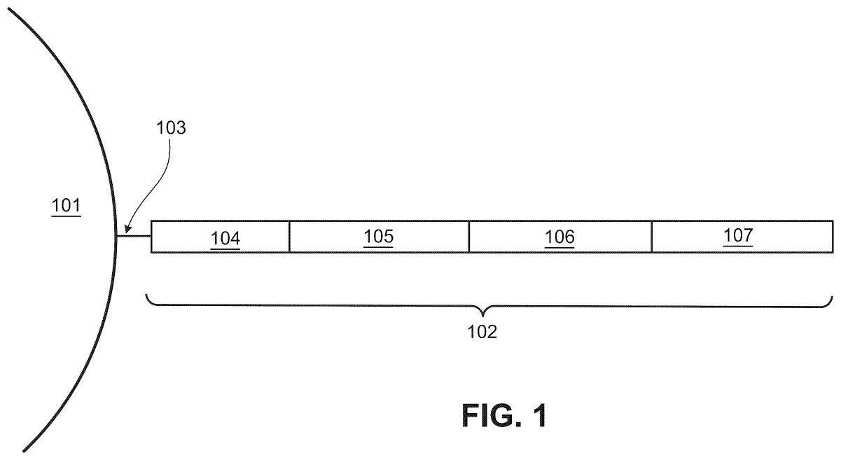

FIG. 1 is a schematic diagram showing an exemplary capture probe, as described herein. As shown, the capture probe 102 is optionally coupled to a feature 101 by a cleavage domain 103, such as a disulfide linker. The capture probe can include a functional sequence 104 that are useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 1 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte. In some embodiments, the capture probe comprises one or more additional functional sequences that can be located, for example between the spatial barcode 105 and the UMI sequence 106, between the UMI sequence 106 and the capture domain 107, or following the capture domain 107. The capture domain can have a sequence complementary to a sequence of a nucleic acid analyte. The capture domain can have a sequence complementary to a ligation product described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence complementary to a sequence of a nucleic acid analyte, a portion of a ligation product described herein, a capture handle sequence described herein, and/or a methylated adaptor described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 105 and functional sequences 104 is common to all of the probes attached to a given feature. In some embodiments, the UMI sequence 106 of a capture probe attached to a given feature is different from the UMI sequence of a different capture probe attached to the given feature.

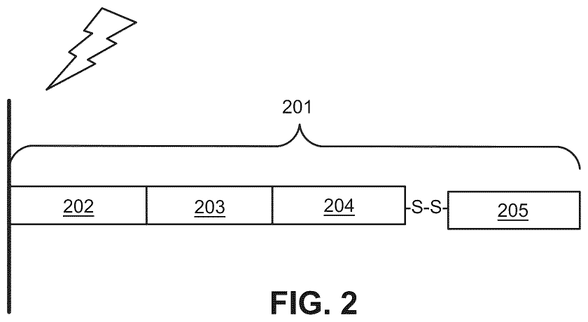

FIG. 2 is a schematic illustrating a cleavable capture probe, wherein the cleaved capture probe can enter into a non-permeabilized cell and bind to analytes within the sample. The capture probe 201 contains a cleavage domain 202, a cell penetrating peptide 203, a reporter molecule 204, and a disulfide bond (—S—S—). 205 represents all other parts of a capture probe, for example a spatial barcode and a capture domain. Cleavable capture probe are further described in WO 2020/176788 and/or U.S. Patent Application Publication No. 2020/0277663, each of which is incorporated by reference in its entirety.

For multiple capture probes that are attached to a common array feature, the one or more spatial barcode sequences of the multiple capture probes can include sequences that are the same for all capture probes coupled to the feature, and/or sequences that are different across all capture probes coupled to the feature.

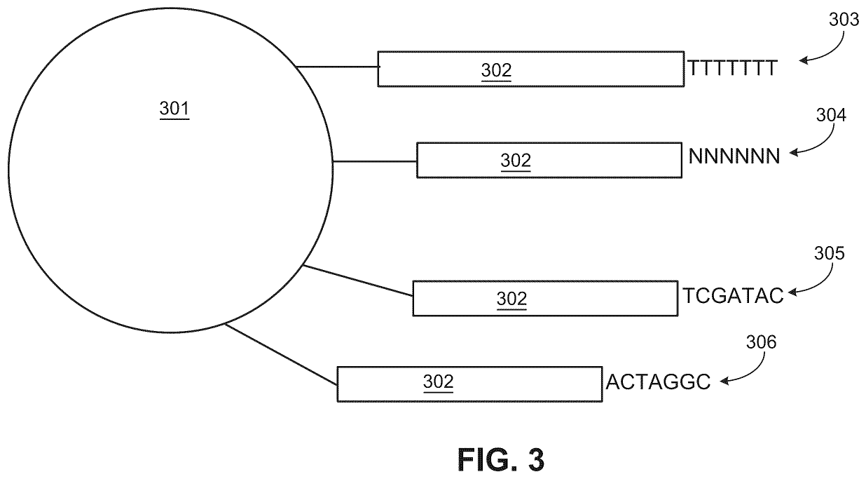

FIG. 3 is a schematic diagram of an exemplary multiplexed spatially-barcoded feature. In FIG. 3, the feature 301 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may be coupled to four different types of spatially-barcoded capture probes, each type of spatially-barcoded capture probe possessing the spatial barcode 302. One type of capture probe associated with the feature includes the spatial barcode 302 in combination with a poly(T) capture domain 303, designed to capture mRNA target analytes. A second type of capture probe associated with the feature includes the spatial barcode 302 in combination with a random N-mer capture domain 304 for gDNA analysis. A third type of capture probe associated with the feature includes the spatial barcode 302 in combination with a capture domain complementary to a capture handle sequence of an analyte capture agent of interest 305. A fourth type of capture probe associated with the feature includes the spatial barcode 302 in combination with a capture domain that can specifically bind a nucleic acid molecule 306 that can function in a CRISPR assay (e.g., CRISPR/Cas9). While only four different capture probe-barcoded constructs are shown in FIG. 3, capture-probe barcoded constructs can be tailored for analyses of any given analyte associated with a nucleic acid and capable of binding with such a construct. For example, the schemes shown in FIG. 3 can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V (D) J sequence of an immune cell receptor (e.g., T-cell receptor). In some embodiments, a perturbation agent can be a small molecule, an antibody, a drug, an aptamer, a miRNA, a physical environmental (e.g., temperature change), or any other known perturbation agents. See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

Capture probes attached to a single array feature can include identical (or common) spatial barcode sequences, different spatial barcode sequences, or a combination of both. Capture probes attached to a feature can include multiple sets of capture probes. Capture probes of a given set can include identical spatial barcode sequences. The identical spatial barcode sequences can be different from spatial barcode sequences of capture probes of another set.

The plurality of capture probes can include spatial barcode sequences (e.g., nucleic acid barcode sequences) that are associated with specific locations on a spatial array. For example, a first plurality of capture probes can be associated with a first region, based on a spatial barcode sequence common to the capture probes within the first region, and a second plurality of capture probes can be associated with a second region, based on a spatial barcode sequence common to the capture probes within the second region. The second region may or may not be associated with the first region. Additional pluralities of capture probes can be associated with spatial barcode sequences common to the capture probes within other regions. In some embodiments, the spatial barcode sequences can be the same across a plurality of capture probe molecules.

In some embodiments, multiple different spatial barcodes are incorporated into a single arrayed capture probe. For example, a mixed but known set of spatial barcode sequences can provide a stronger address or attribution of the spatial barcodes to a given spot or location, by providing duplicate or independent confirmation of the identity of the location. In some embodiments, the multiple spatial barcodes represent increasing specificity of the location of the particular array point.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence or capture handle sequence. As used herein, the term “analyte binding moiety barcode” refers to a barcode that is associated with or otherwise identifies the analyte binding moiety. As used herein, the term “analyte capture sequence” or “capture handle sequence” refers to a region or moiety configured to hybridize to, bind to, couple to, or otherwise interact with a capture domain of a capture probe.

In some embodiments, a capture handle sequence is complementary to a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of WO 2020/176788 and/or Section (II)(b)(viii) U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA template, such as an analyte or an intermediate agent (e.g., a ligation product or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form ligation products with a template (e.g., a DNA template, such as an analyte or an intermediate agent, or portion thereof), thereby creating ligations products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

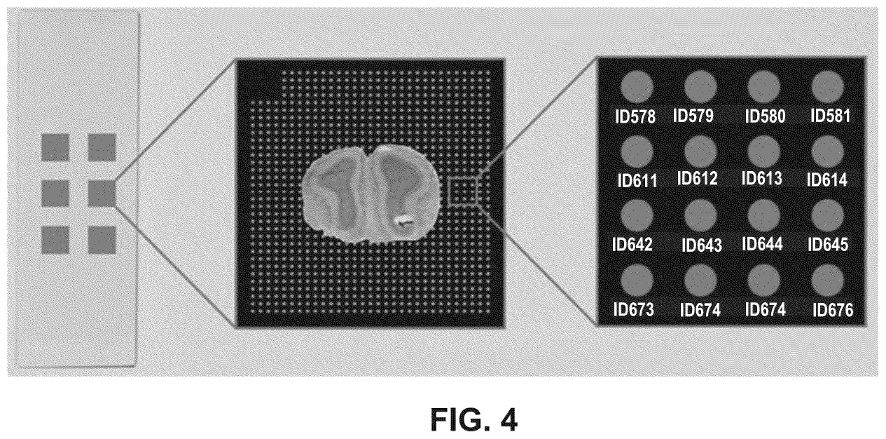

FIG. 4 depicts an exemplary arrangement of barcoded features within an array. From left to right, FIG. 4 shows (L) a slide including six spatially-barcoded arrays, (C) an enlarged schematic of one of the six spatially-barcoded arrays, showing a grid of barcoded features in relation to a biological sample, and (R) an enlarged schematic of one section of an array, showing the specific identification of multiple features within the array (labelled as ID578, ID579, ID560, etc.).

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion).

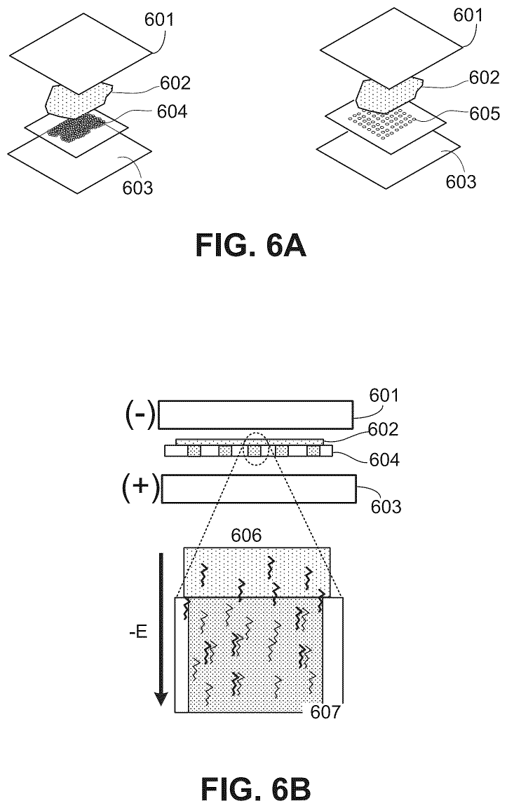

Schematics illustrating an electrophoretic transfer system configured to direct nucleic acid analytes (e.g., mRNA transcripts) toward a spatially-barcoded capture probe array are shown in FIG. 6A and FIG. 6B. In this exemplary configuration of an electrophoretic system, a sample 602 is sandwiched between the cathode 601 and the spatially-barcoded capture probe array 604, 605, and the spatially-barcoded capture probe array 604, 605 is sandwiched between the sample 602 and the anode 603, such that the sample 602, 606 is in contact with the spatially-barcoded capture probes 607. When an electric field is applied to the electrophoretic transfer system, negatively charged nucleic acid analytes 606 will be pulled toward the positively charged anode 603 and into the spatially-barcoded array 604, 605 containing the spatially-barcoded capture probes 607. The spatially-barcoded capture probes 607 interact with the nucleic acid analytes (e.g., mRNA transcripts hybridize to spatially-barcoded nucleic acid capture probes forming DNA/RNA hybrids) 606, making the analyte capture more efficient. The electrophoretic system set-up may change depending on the target analyte. For example, proteins may be positive, negative, neutral, or polar depending on the protein as well as other factors (e.g., isoelectric point, solubility, etc.). The skilled practitioner has the knowledge and experience to arrange the electrophoretic transfer system to facilitate capture of a particular target analyte.

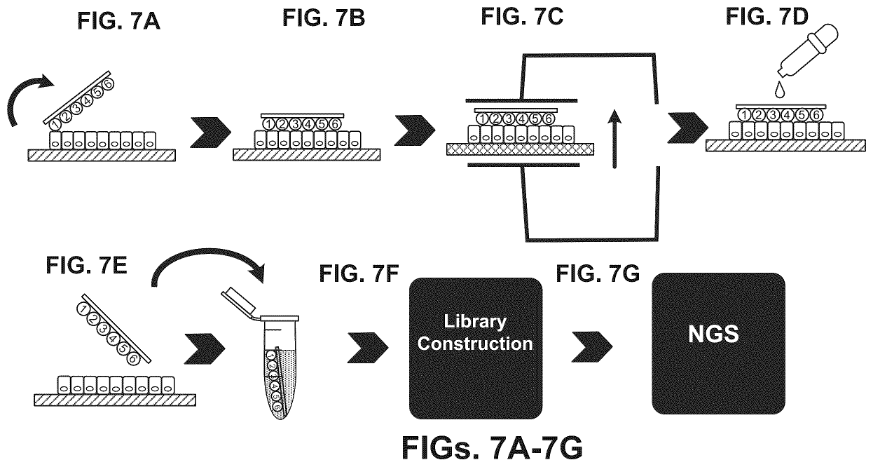

FIGS. 7A-7G are illustrations showing an exemplary workflow protocol utilizing an electrophoretic transfer system. In the example, FIG. 7A depicts a flexible spatially-barcoded feature array being contacted with a sample. The sample can be a flexible array, wherein the array is immobilized on a hydrogel, membrane, or other flexible substrate. FIG. 7B depicts contact of the array with the sample and imaging of the array-sample assembly. The image of the sample/array assembly can be used to verify sample placement, choose a region of interest, or any other reason for imaging a sample on an array as described herein. FIG. 7C depicts application of an electric field using an electrophoretic transfer system to aid in efficient capture of a target analyte. Here, negatively charged mRNA target analytes migrate toward the positively charged anode. FIG. 7D depicts application of reverse transcription reagents and first strand cDNA synthesis of the captured target analytes. FIG. 7E depicts array removal and preparation for library construction (FIG. 7F) and next-generation sequencing (FIG. 7G).

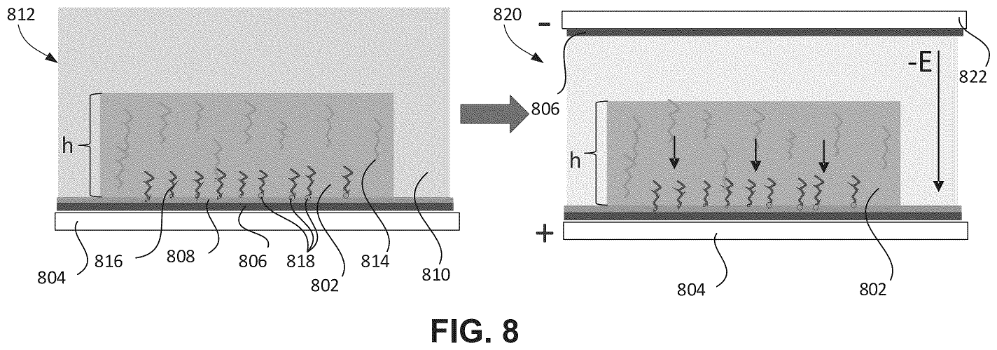

FIG. 8 shows an example of an analytical workflows for active capture methods using an electric field (e.g., using electrophoresis). In some examples, a biological sample 802 (e.g., a tissue sample) can be in contact with a first substrate 804. In some embodiments, first substrate 804 can have one or more coatings (e.g., any of the conductive substrates described herein) on its surface. Non-limiting examples of coatings include, nucleic acids (e.g., RNA) and conductive oxides (e.g., indium tin oxide). In some embodiments, first substrate 804 can have a functionalization chemistry on its surface. In the example shown in FIG. 8, first substrate 804 is overlaid with a first coating 806, and first coating 806 (e.g., a conductive coating) is further overlaid with a second coating 808. In some embodiments, first coating 806 is an indium tin oxide (ITO) coating. In some embodiments, second coating 808 is a lawn of capture probes (e.g., any of the capture probes described herein). In some embodiments, a substrate can include an ITO coating. In some embodiments, a substrate can include capture probes or capture probes attached to features on the substrate.

Biological sample 802 and second coating 808 (e.g., a lawn of capture probes) can be in contact with a permeabilization solution 810. Non-limiting examples of permeabilization solutions include, enzymes (e.g., proteinase K, pepsin, and collagenase), detergents (e.g., sodium dodecyl sulfate (SDS), polyethylene glycol tert-octylphenyl ether, polysorbate 80, and polysorbate 20), ribonuclease inhibitors, buffers optimized for electrophoresis, buffers optimized for permeabilization, buffers optimized for hybridization, or combinations thereof. Permeabilization reagents can also include but are not limited to a dried permeabilization reagent, a permeabilization buffer, a buffer without a permeabilization reagent, a permeabilization gel, and a permeabilization solution. In some examples, biological samples (e.g., tissue samples) can be permeabilized first and then be subjected to electrophoresis.

FIG. 8 shows an example analytical workflow including a first step 812 in which biological sample 802 can be permeabilized prior to subjecting the sample 802 to electrophoresis. Any of the permeabilization methods disclosed herein can be used during first step 812. Biological sample 802 includes an analyte 814. In some embodiments, the analyte 814 is a negatively charged analyte. First substrate 804 can include a capture probe 816 that is fixed or attached to the first substrate 804 or attached to features (e.g., beads) 818 on the substrate. In some embodiments, capture probe 816 can include any of the capture probes disclosed herein. In some embodiments, first substrate 802 does not include features and instead, capture probes 816 are directly attached to the substrate surface. In some embodiments, the capture probe 816 is positively charged.

In step 820, after permeabilization of biological sample 802 concludes, the sample 802 can be subjected to electrophoresis. During electrophoresis, the biological sample 802 is subjected to an electric field that can be generated by sandwiching biological sample 802 between the first substrate 804 and a second substrate 822, connecting each substrate to a cathode and an anode, respectively, and running an electric current through the substrates. The application of the electric field “-E” causes the analyte 804 (e.g., a negatively charged analyte) to migrate towards the substrate 804 and capture probe 816 (e.g., a positively charged capture probe) in the direction of the arrows shown in FIG. 8. In some embodiments, the analyte 814 migrates towards the capture probe 816 for a distance “h.” In some embodiments, the analyte 814 migrates towards a capture probe 816 through one or more permeabilized cells within the permeabilized biological sample (e.g., from an original location in a permeabilized cell to a final location in or close to the capture probe 816). Second substrate 822 can include the first coating 806 (e.g., a conductive coating), thereby allowing electric field “-E” to be generated.

In some embodiments, the analyte 814 is a protein or a nucleic acid. In some embodiments, the analyte 814 is a negatively charged protein or a nucleic acid. In some embodiments, the analyte 814 is a positively charged protein or a nucleic acid. In some embodiments, the capture probe 816 is a protein or a nucleic acid. In some embodiments, the capture probe 816 is a positively charged protein or a nucleic acid. In some embodiments, the capture probe 816 is a negatively charged protein or a nucleic acid. In some embodiments, the analyte 814 is a negatively charged transcript. In some embodiments, the analyte 814 is a poly(A) transcript. In some embodiments, the capture probe 816 is attached to a feature in a feature array. In some embodiments, permeabilization reagent 810 can be in contact with sample 802, first substrate 804 second substrate 822, or any combination thereof.

Alternatively, biological sample 802 can be permeabilized and subjected to electrophoresis simultaneously. In some embodiments, simultaneous permeabilization and electrophoresis of biological sample 802 can decrease the total duration of the analytical workflow translating into a more efficient workflow.

In some embodiments, the permeabilization reaction is conducted at a chilled temperature (e.g., about 4° C.). In some embodiments, conducting the permeabilization reaction at a chilled temperature controls the enzyme activity of the permeabilization reaction. In some embodiments, the permeabilization reaction is conducted at a chilled temperature in order to prevent drift and/or diffusion of the analyte 814 from an original location (e.g., a location in a cell of the biological sample 802) until a user is ready to initiate the permeabilization reaction. In some embodiments, the permeabilization reaction is conducted at a warm temperature (e.g., a temperature ranging from about 15° C. to about 37° C. or more) in order to initiate and/or increase the rate of the permeabilization reaction. In some embodiments, once electrophoresis is applied and/or the permeabilization reaction is heated, the permeabilization reaction allows for analyte migration from an original location (e.g., a location in a cell of the biological sample 802) to the capture probe 816 anchored to the first substrate 804.

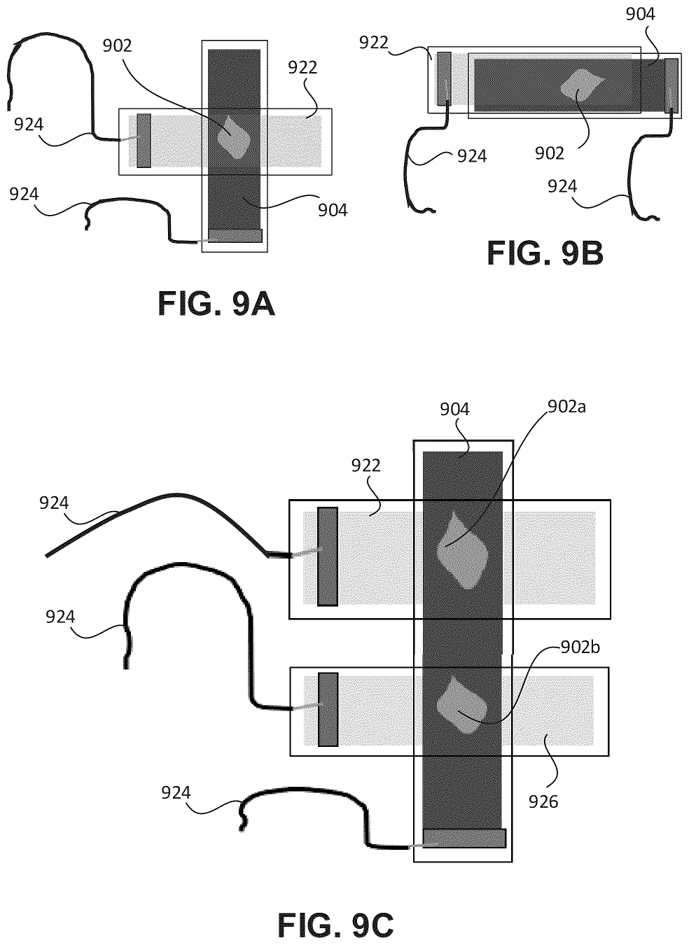

Referring generally to FIGS. 9A-9C, example substrate configurations for use in the active migration of analytes from a first location to a second location via electrophoresis are shown. FIG. 9A shows an example substrate configuration for use in electrophoresis in which the first substrate 904 and the second substrate 922 are aligned at about 90 degrees with respect to each other. In this example, the first substrate 904 including biological sample 902 is placed beneath second substrate 922. Both the first substrate 904 and the second substrate 922 can be connected to electrical wires 924 that direct an electric current from a power supply to the substrates, thereby generating an electric field between the substrates. FIG. 9B shows an additional example substrate configuration for use during electrophoresis in which the first substrate 904 and the second substrate 922 are parallel with respect to each other. In this example, the first substrate 904 including biological sample 902 is also placed beneath second substrate 922.

FIG. 9C shows yet an additional example substrate configuration for use in electrophoresis in which the second substrate 922 and a third substrate 926 are aligned at about 90 degrees with respect to the first substrate 904. Thus, in this example, a first biological sample 902a and a second biological sample 902b can be subjected to electrophoresis simultaneously. In some embodiments, 3, 4, 5, 6, 7, 8, 9, 10, or more biological samples can be placed on a same substrate and be subjected to electrophoresis simultaneously. In some embodiments, 2, 3, 4, 5, 6, 7, 8, 9, 10, or more top substrates can be placed above a same bottom substrate containing one or more samples in order to simultaneously subject the one or more samples to electrophoresis. In some embodiments, 2, 3, 4, 5, 6, 7, 8, 9, 10, or more top substrates can be perpendicularly placed (e.g., at about 90 degrees) above a same bottom substrate containing one or more samples in order to simultaneously subject the one or more samples to electrophoresis. In some embodiments, 2, 3, 4, 5, 6, 7, 8, 9, 10, or more top substrates can be placed in a parallel orientation above a same bottom substrate containing one or more biological samples in order to simultaneously subject the one or more samples to electrophoresis. In some embodiments, a configuration of top substrates can be arranged above a same bottom substrate containing one or more biological samples in order to simultaneously subject the one or more samples to electrophoresis. In some embodiments, a first configuration of top substrates can be arranged above a second array of bottom substrates containing one or more biological samples in order to simultaneously subject the one or more biological samples to electrophoresis. In some embodiments, simultaneously subjecting two or more biological samples on a same substrate to electrophoresis can provide the advantage of a more effective workflow. In some embodiments, one or more of the top substrates can contain the biological sample.

Analyte capture is further described in Section (II)(e) of WO 2020/176788 and/or U.S. Patent Application Publication No. 2020/0277663.

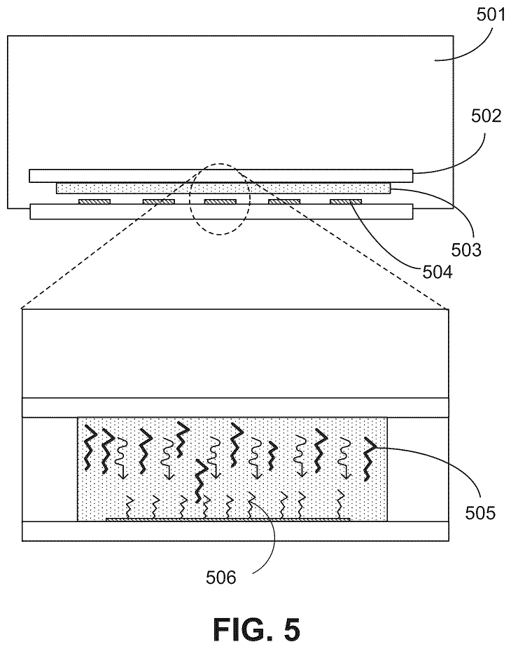

FIG. 5 is an illustration of an exemplary use of a diffusion-resistant medium. A diffusion-resistant medium 502 can be contacted with a sample 503. In FIG. 5, a glass slide 504 is populated with spatially-barcoded capture probes 506, and the sample 503, 505 is contacted with the array 504, 506. A diffusion-resistant medium 502 can be applied to the sample 503, wherein the sample 503 is disposed between a diffusion-resistant medium 502 and a capture probe coated slide 504. When a permeabilization solution 501 is applied to the sample, the diffusion-resistant medium 502 facilitates the migration of the analytes 505 toward proximal capture probes 506 by reducing diffusion of the analytes out into the medium. Alternatively, the diffusion resistant medium may include permeabilization reagents.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14):e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNAse H). The released ligation product can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application No. 2020/061064 and/or U.S. patent application Ser. No. 16/951,854.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2020/061108 and/or U.S. patent application Ser. No. 16/951,864.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application No. 2020/061066, and/or U.S. patent application Ser. No. 16/951,843. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distance.

### II. Method and Compositions for Spatial Analysis by Isolating Regions of Interest Using Hydrogels

Provided herein are methods for identifying (e.g., determining the abundance and location of) one or more analytes in a region of interest from a biological sample. It has been identified that processing an entire biological sample can result in increased data (e.g., sequencing) in regions of the biological sample that might not be of interest.

In some instances, the methods include identifying a region of interest of the biological sample. Selecting a region of interest (or multiple regions of interest) to process rather than processing the entire biological sample eliminates unnecessary downstream processing (e.g., sequencing) of regions that are not of interest, which provides cost, resource, and time savings. Further, because the methods herein include determination of the presence of analytes in a region of interest, the methods provide a resolution of analytes in the region. Selecting a region of interest such as a region identified by a pathologist as including a tumor allows for focused detection and understanding of the heterogeneity of a sample. In some instances, heterogeneity can be studied by comparing an analyte in the region of interest to the other analytes in the region; or in some instances, the analyte can be compared to its expression throughout the sample. Thus, the methods disclosed herein provide information on the analyte spatial heterogeneity of a sample.

### A. Spatial Tagging Regions of Interests Using a Hydrogel

The disclosure provides methods and compositions for capturing one or more analytes from a biological sample using a hydrogel. A “hydrogel” as described herein can include a cross-linked three-dimensional network of hydrophilic polymer chains. In some instances, a hydrogel comprises one or more hydrogel subunits. A “hydrogel subunit” can be a hydrophilic monomer, a molecular precursor, or a polymer that can be polymerized (e.g., cross-linked) to form a three-dimensional (3D) hydrogel network. The hydrogel forms a layer either directly on the substrate or over the biological sample, which is placed on the substrate. As discussed herein, the hydrogel serves as a barrier between the analyte and a capture probe that disallows analyte capture at regions that include the hydrogel. In some instances, the hydrogel is removed at certain regions (e.g., at regions of interest) to allow for spatial analysis of analytes at these regions.

In some embodiments, a hydrogel comprises a natural material. In some embodiments, a hydrogel includes a synthetic material. In some embodiments, a hydrogel includes a hybrid material, e.g., the hydrogel material comprises elements of both synthetic and natural polymers. Any of the materials used in hydrogels or hydrogels comprising a polypeptide-based material described herein can be used. Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other hydrogel-formation method known in the art. For example, the biological sample can be immobilized in the hydrogel by polyacrylamide crosslinking. Further, analytes of a biological sample can be immobilized in a hydrogel by crosslinking (e.g., polyacrylamide crosslinking).

A hydrogel can be any appropriate hydrogel where upon formation of the hydrogel the capture probe becomes anchored to or embedded in the hydrogel or the biological sample becomes anchored to, covered, surrounded or embedded in the hydrogel. A hydrogel can include hydrogel subunits. Non-limiting examples of hydrogel subunits include: acrylamide, bis-acrylamide, polyacrylamide and derivatives thereof, poly(ethylene glycol) and derivatives thereof (e.g., PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, or combinations thereof. In some embodiments, the hydrogel is a dithiothreitol (DTT)-sensitive hydrogel.

In some embodiments, the hydrogel is between approximately 5-500 micrometers thick including 500, 450, 400, 350, 300, 250, 200, 150, 100, 50, 20, 10 or 5 micrometers thick, or any thickness within 5 and 500 micrometers. In some embodiments, a “thin hydrogel layer” includes a hydrogel below about 100 micrometers thick (e.g., below about 90 micrometers, below about 80 micrometers, below about 70 micrometers, below about 60 micrometers, below about 50 micrometers, below about 40 micrometers, below about 30 micrometers, below about 20 micrometers, or below about 10 micrometers).

In some embodiments where the method includes degrading a portion of the hydrogel corresponding to a region of interest of the biological sample, the method includes exposing the region of interest to a degrading agent. In some embodiments, the hydrogel is exposed to the degrading agent prior to the substrate contacting a biological sample. Alternatively, the hydrogel is contacted with a biological sample prior to the degradation reagent being deposited on the biological sample and/or the hydrogel, depending on the orientation of the biological sample and the substrate. In some embodiments, degrading the portion of hydrogel includes exposing the region of interest of the biological sample to a degradation agent, where the degradation agent degrades the hydrogel (e.g., underneath the biological sample).

In some embodiments, the degradation agent includes an agent that degrades the hydrogel. For example, the degradation agent is dithiothreitol (DTT). The degradation agent can be in a solution. The solution can include additional reagents, for example, without limitation, a permeabilization reagent, an analyte binding moiety, an oligonucleotide, and a cell-tagging agent, or any combination thereof. In some embodiments, the degradation reagent is provided to the biological sample using a low volume liquid dispenser. Non-limiting examples of low volume liquid dispensing instruments include SCIENION's sciDrop PICO, LabCyte's Echo 525, PolyPico Technologies PicoSpotter, and BioFluidix PicoDispenser.

In some embodiments, degrading the portion of hydrogel that corresponds to the region of interest includes exposing the portion of hydrogel to UV light, laser light, natural light, a heating device, a radiation device, a plasma device, a microwave device or a degradation agent, or any combination thereof.

In some embodiments, degrading the portion of hydrogel that corresponds to the region of interest of the biological sample includes dissolving the portion of hydrogel. In some embodiments, the portion of hydrogel that corresponds to the region of interest is dissolved using DTT. For example, treatment of a DTT-sensitive region of interest in the hydrogel with DTT degrades the DTT sensitive region, thus the capture probes are no longer blocked from interacting with analytes in the biological sample.

In some embodiments, the hydrogel can be any hydrogel as described herein. In some embodiments, the hydrogel can include reagents, including, without limitation, capture probes, permeabilization reagents, nucleic acid extension or ligation reaction reagents, and sequencing library preparation reagents, or any combination thereof.

In some embodiments where the method includes capturing analytes from a biological sample with a hydrogel that includes a plurality of capture probes, the capture probes can be encapsulated within, embedded within, or layered on a surface of a hydrogel. Where a hydrogel includes a structure, a plurality of capture probes can be attached to and/or associated with that structure. Non-limiting examples of structures that can be included on or within a hydrogel include: a well, a nanowell, a depression, a channel, a fiducial mark, a feature, a bead, an analyte-binding moiety (e.g., a protein or an antibody), and an oligonucleotide, or any combination thereof. In some embodiments, a nanowell is printed on the hydrogel. In some embodiments where the hydrogel includes one or more nanowells, the plurality of capture probes can be layered in and/or on the nanowells. In some embodiments, a capture probe is attached to a feature and the feature is encapsulated within, embedded within, or layered on a surface of a hydrogel.

In some instances, capture probes are printed onto the hydrogel. The capture probes, as described here, include a capture domain that includes a sequence (e.g., an oligo d (T) sequence) that is fully or partially complementary to a sequence of an analyte (e.g., a poly(A) tail for mRNA capture) or an analyte-binding moiety (e.g., a capture sequence specific for a barcoded antibody). In some embodiments, a capture probe is embedded in a hydrogel to facilitate capture of analytes from the biological sample. For example, where one or more regions of interest are identified, a hydrogel including capture probes embedded in the hydrogel can be used to selectively isolate and transfer the portion of the hydrogel that corresponds to the region of interest of the biological sample. In some cases, the hydrogel serves as a barrier to prevent interaction between capture probes and analytes in a biological sample. In some embodiments, a plurality of capture probes can be deposited with any of the prepolymer solutions described herein. In some embodiments, the prepolymer solution can be polymerized such that a hydrogel is formed around the plurality of capture probes. Hydrogel formation can occur in a manner sufficient to anchor (e.g., embed) the capture probes in the hydrogel. For example, a capture probe can be anchored to the hydrogel at its 5′ end. In another example, the capture probe is anchored to the hydrogel at its 3′ end. After hydrogel formation, the capture probe is anchored to (e.g., embedded in) the hydrogel wherein the hydrogel can be used for spatial tagging of analytes in a biological sample. In some embodiments, the capture probe is anchored to the hydrogel via a suitable linker.

In some cases, a hydrogel includes capture probes encapsulated, embedded in, or layered on the surface of the hydrogel. The hydrogel can be any shape that allows capture of an analyte by the capture probes embedded in the hydrogel. In some cases, the hydrogel is a sheet, a slab, a partition, or any other form that enables capture of analytes by the encapsulated, embedded or layered capture probes. The hydrogel can be flat (e.g., planar) relative to the biological sample to which it is contacted. Alternatively, the hydrogel can be convex or concave relative to the biological sample. The hydrogel can be prepared as a regular shaped (e.g., square, rectangle or oval) or polygon hydrogel.

In some cases, to form a hydrogel where the capture probes are embedded in the hydrogel, a hydrogel can be formed around capture probes that are reversibly fixed to the surface of a substrate. In such cases, the capture probes can be released prior to, contemporaneously with, or after hydrogel formation. In some cases where the hydrogel includes nanowells imprinted into the hydrogel, the capture probes are printed onto one or more surfaces of the nanowells.

The hydrogel that includes the capture probes can be contacted with a biological sample. In such cases, the hydrogel can be contacted with a biological sample in any manner that facilitates capture of the analytes by the capture probes. In some embodiments, a region of interest of the biological sample is identified before, contemporaneously with, of after, the hydrogel is contacted with the biological sample. Permeabilization reagents can be applied to the one or more regions of interest to facilitate capture of analytes from the biological sample. In some instances, the permeabilization reagent comprises pepsin or proteinase K.

In some instances, after the capture probes are affixed to the hydrogel, a biological sample is placed on the hydrogel. After permeabilization of the biological sample using methods disclosed herein, analytes of the biological sample are captured by capture probes on the hydrogel. In some instances, one or more (e.g., at least one, at least 2, at least 3, at least, 4, at least 5, or more) regions of interest in the biological sample are identified. A region of interest as used herein is a subsection (e.g., a sub-area) of a biological sample that has been identified as having certain characteristics of interest. For example, a region of interest can include a region of a biological sample comprising a tumor. In some instances, the region of interest is a non-tumor area of the biological sample. In some instances, the region of interest includes a region that includes fibrous tissue, immune cell infiltrates, expression of cellular markers of interest, or any combination thereof. In some instances, one or more regions of interest can be present within a single biological sample (e.g., a tissue section), each region of interest having the same or different dimensions (e.g., size, cell number, area, etc.). It is appreciated that a user can determine a region of interest using many different parameters.

In some instances, the methods include providing a substrate including a hydrogel layered over a plurality of capture probes to capture the analyte, identifying a region of interest of the biological sample, degrading the hydrogel, and hybridizing an analyte from the region of interest to the capture domain located within the portion of the substrate that corresponds to the region of interest of the biological sample.

In a non-limiting example, this disclosure provides a method for determining a location and/or an abundance of an analyte in a region of interest from a biological sample that includes: (a) contacting the biological sample with a substrate including a hydrogel layered over a plurality of capture probes, wherein a capture probe of the plurality of capture probes includes a spatial barcode and a capture domain; (b) identifying the region of interest of the biological sample; (c) degrading a portion of hydrogel corresponding to the region of interest of the biological sample, thereby exposing the capture probe to the biological sample; (d) hybridizing the analyte to the capture domain at the region of interest; (e) determining (i) all or a portion of a sequence of the spatial barcode, or a complement thereof, and (ii) all or a portion of a sequence of the analyte, or a complement thereof, and using the sequences of (i) and (ii) to identify the location of the analyte in the biological sample.

In another non-limiting example, the disclosure provides a method of determining a location and/or an abundance of an analyte in a region of interest from a biological sample that includes: (a) aligning the biological sample with a substrate including a hydrogel layered over a plurality of capture probes, wherein a capture probe of the plurality of capture probes includes a spatial barcode and a capture domain; (b) identifying the region of interest of the biological sample; (c) degrading a portion of hydrogel corresponding to the region of interest of the biological sample, thereby exposing the capture probes in that portion; (d) contacting the biological sample with the substrate, thereby allowing the analytes present in the region of interest to hybridize to the capture probes; and (e) determining (i) all or a portion of a sequence of the spatial barcode, or a complement thereof, and (ii) all or a portion of a sequence of the analyte, or a complement thereof, and using the sequences of (i) and (ii) to identify the location of the analyte in the biological sample.

In some cases, a substrate includes a hydrogel layered over a plurality of capture probes. A portion of the hydrogel corresponding to the region of interest of a biological sample can be degraded thereby exposing only the capture probes at the region of interest. A portion of the hydrogel can be degraded before, contemporaneously with, or after contacting the substrate with a biological sample. In cases where a portion of the hydrogel is degraded before contacting the substrate with the biological sample, the biological sample is aligned with the substrate prior to degrading the portion of hydrogel. In some cases, a region of interest of the biological sample is identified prior to aligning the substrate with the biological sample. In some cases, a region of interest of the biological sample is identified after aligning the substrate with the biological sample. In some cases, a portion of the hydrogel layer can be degraded using laser light or UV light. The biological sample is then contacted with the substrate having one or more portions of the hydrogel degraded, thereby exposing the capture probes in those area. The one or more portions of the hydrogel that are degraded correspond to the regions of interest in the biological sample.

In cases where a portion of the hydrogel is to be degraded after contacting the substrate with a biological sample, a region of interest of the biological sample can be identified before, contemporaneously with, or after contacting the substrate with the biological sample. In such cases, the biological sample is contacted with the substrate in a manner that avoids damaging the hydrogel. Damaging the hydrogel in this instance could expose the biological sample to the capture probes on the substrate and result in analyte capture from areas of the hydrogel that are not considered regions of interest.

In such cases where the biological sample and the substrate are contacted prior to degradation of the portion of the hydrogel, the portion of the hydrogel can be degraded using laser light of UV light. Alternatively, the portion of the hydrogel that correspond to the region of interest in the biological sample is degraded using a degrading agent (e.g., any of the degrading agents described herein). In such cases, the degrading agent permeates the biological sample before degrading the portion of the hydrogel underneath the region of interest of the biological sample.

In cases where the portion of the hydrogel is degraded before contacting the substrate with the biological sample and in cases where the portion of the hydrogel is degraded after contacting the substrate with the biological sample, the analytes from the regions of interest in the biological sample hybridize to the capture probes on the substrate. Following hybridization the biological sample can be removed from the substrate (e.g., physically or enzymatically). The remainder of the hydrogel can be removed either using laser ablation or a chemical degradation agent. The substrate can then be processed according to the methods described herein (e.g., capture probe extension, second strand cDNA synthesis, amplification, or any combination thereof). Alternatively, only the capture probes corresponding to the region of interest in the biological sample are processed further. For example, the capture probes corresponding to the region of interest are processed (e.g., capture probe extension, second strand cDNA synthesis, amplification, or any combination thereof) while the remainder of the capture probes affixed to the surface of the substrate remain blocked by hydrogel. If a capture probe remains blocked by hydrogel or within the hydrogel, the capture probe is blocked or substantially reduced from interacting with an analyte (e.g., hybridizing to an analyte) and is not processed according to the methods described herein.

### B. Identifying a Region of Interest

In some embodiments, determining a location and/or an abundance of a region of interest can include interrogating a biological sample using a variety of different techniques, e.g., expansion microscopy, bright field microscopy, dark field microscopy, phase contrast microscopy, electron microscopy, fluorescence microscopy, reflection microscopy, interference microscopy, confocal microscopy, electrophysiology (e.g., patch clamping or sharp electrode) and visual identification (e.g., by eye), or combinations thereof. For example, a region of interest can be identified by staining and imaging of the biological sample.

In some instances, the methods disclosed herein include providing capture probes to capture the analyte, identifying a region of interest of a biological sample, and isolating the region of interest of the biological sample. A “region of interest” as used herein refers to a subsection of a biological sample (e.g., a subsection of a tissue section) that is identified for further spatial analysis. In some instances, the region of interest corresponds to expression of a particular stain (e.g., an H&E stain, an immunofluorescence stain; an immunohistochemistry stain). In some instances, the region of interest corresponds to expression of a particular analyte (e.g., a protein or nucleic acid) at that subsection of the sample. In some instances, the region of interest corresponds to a subsection of a tissue comprising one or more pathophysiological features, such as a tumor or an area comprising immune cell infiltrates. It is appreciated that a user can define the area, including the size and shape, of the region of interest using any method known in the art.

In some instances, the region of interest is isolated by removing the portion of the hydrogel that corresponds to the region of interest of the biological sample using mechanical transfer (e.g., a mechanical hole punch, microsurgery, laser capture microdissection, or a combination thereof). In a non-limiting example, this disclosure provides a method of identifying an analyte in a region of interest from a biological sample that includes: (a) contacting the biological sample with a hydrogel including a plurality of capture probes, wherein a capture probe of the plurality of capture probes includes a spatial barcode and a capture domain; (b) hybridizing the analyte to the capture domain; (c) identifying the region of interest of the biological sample; (d) isolating the region of interest of the biological sample; and (e) determining (i) all or a portion of a sequence of the spatial barcode, or a complement thereof, and (ii) all or a portion of a sequence of the analyte, or a complement thereof, and using the sequences of (i) and (ii) to identify the analyte in a region of interest in the biological sample. In some embodiments, the method includes extending the capture probe and generating second strand cDNA prior to the isolating step. In some embodiments, the determining step includes amplifying all or part of the second strand cDNA bound to the capture probe. Amplification can include isothermal or non-isothermal amplification.

In some embodiments, the method includes isolating the region of interest of the biological sample by removing the portion of the hydrogel that corresponds to the region of interest of the biological sample using mechanical transfer. Non-limiting examples of mechanical transfer include mechanical hole punch, microsurgery, or laser capture microdissection (LCM), or a combination thereof. In some embodiments, the mechanical transfer includes cutting the region of interest from the biological sample. In some instances, the mechanical transfer includes using a mechanical stencil or scalpel to cut the region of interest from the biological sample. In some embodiments, the mechanical transfer includes LCM.

In some embodiments, isolating the region of interest of the biological sample includes degrading the hydrogel. In some instances, degrading the hydrogel releases the analytes for downstream analysis. In some embodiments, the method includes degrading the hydrogel after the isolating step under conditions that prevent degradation of the analyte. In some embodiments, the hydrogel is degraded using a reducing agent. In some instances, the reducing agent is dithiothreitol (DTT). In some embodiments, the hydrogel includes a reagent to prevent the degradation of the analyte. In some embodiments, the reagent includes an RNase or DNase inhibitor.

In some cases, the capture probes corresponding to a region of interest are processed (e.g., capture probe extension, second strand cDNA synthesis, amplification, or any combination thereof) while the remainder of the capture probes affixed to the surface of the substrate remain blocked by hydrogel. In some cases, more than just the capture probes corresponding to the region of interest are processed (e.g., capture probe extension, second strand cDNA synthesis, amplification, or any combination thereof). In some cases, following capture of the analyte, no additional processing is performed until after the region of interest of the biological sample is isolated.

In some cases, a region of interest of the biological sample identified by any of exemplary means described herein can be isolated using mechanical transfer (e.g., any of the mechanical transfer methods described herein). In some cases, automation of any of the steps in any of the methods described herein can help to increase efficiency of analyte capture.

In some embodiments, a region of interest is identified based on a measured signal. In some embodiments, the biological sample is stained using a detectable label. For example, following staining of the biological sample using a detectable label, the detectable label identifies a region of interest. In some embodiments, a biological sample can be stained using immunofluorescence or immunohistochemistry. In some embodiments, the detectable label is Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS).

In some embodiments, a region of interest is identified based on a morphological characteristic of the biological sample (e.g., hippocampus, hypothalamus, tumor cells or immune cell infiltrate).

In some embodiments, the method includes isolating at least 2, at least 3, at least 4, at least 5, or more regions of interest in the biological sample (e.g., a single tissue section).

In some embodiments, once the region of interest has been identified, the region of interest is assigned a location (e.g., an x,y coordinate). For example, when a region of interest is identified using bright field microscopy, an image of the region of interest can be taken and assigned an x,y coordinate. This x,y coordinate can then be used as the location for depositing, for example, a capture probe and a permeabilization reagent. In some instances, the permeabilization reagent comprises pepsin or proteinase K. In some embodiments, an image registration system is used to mark or track the location of the image of the region of interest. The image registration system can then be used to direct the depositing of, for example, a capture probe and a permeabilization reagent onto the region of interest. In some embodiments, fiducial markers can be used to register the location of the region of interest within the biological sample. For example, the region of interest can be assigned a location based on its relative position to a fiducial marker where the relative position to the fiducial marker can be used to direct the depositing of, for example, a capture probe and a permeabilization reagent onto the region of interest.

In some embodiments, the method can also include aligning the biological sample and the substrate prior to transferring the analyte (e.g., RNA) from the biological sample to the substrate. In some embodiments, aligning the biological sample and the substrate occurs after the region of interest has been identified. In some embodiments, the alignment is recorded as a location, where each of the biological sample and the substrate are assigned a location (e.g., an x,y coordinate).

In some instances, areas outside the region of interest can be stored for later use. In some instances, areas outside the region of interest can be isolated from the region of interest and analyzed using methods disclosed herein. For instance, capture probes outside of the area of interest can detect analytes outside of the area of interest using the same methods of analyte detection (e.g., hybridization, capture probes extension) that are used for capture probes that hybridize to analytes in the area of interest. In some instances, analytes in areas outside the region of interest can be compared to analytes in the region of interest. In some instances, analytes in areas outside the region of interest are discarded or do not undergo further processing (e.g., sequencing).

Also provided herein are laser capture microdissection methods used for identifying an analyte in a region of interest from a biological sample. In some instances, the method includes identifying a region of interest of the biological sample and using laser capture microdissection or laser ablation methods to select a region of interest to process rather than processing the entire biological sample (e.g., a whole tissue section). As mentioned above, this eliminates unnecessary downstream processing of regions that are not of interest, which provides cost, resource and time savings.

In some instances, laser capture microdissection includes positive selection where a region of interest or portion of the biological sample is “cut” out. In some instances, laser capture microdissection includes negative selection where laser ablation eliminates portions of the biological sample not associated with a region of interest. Laser capture microdissection is described further in Espina et al., Nature Protocols, 1, 586-603 (2006), which is incorporated by reference in its entirety. In some embodiments, the methods provided herein include the use of laser light to ablate regions of the biological sample that are not of interest. In some embodiments, the methods provided herein include the use of laser light to isolate a region of interest of the biological sample. In some embodiments, the methods provided herein include the use of laser light to degrade a portion of a hydrogel. In some embodiments, laser light is used to degrade a portion of a hydrogel corresponding to a region of interest in a biological sample.

In a non-limiting example, this disclosure provides a method of identifying an analyte in a region of interest from a biological sample that includes: (a) contacting the biological sample with a hydrogel including a plurality of capture probes, wherein a capture probe of the plurality of capture probes includes a spatial barcode and a capture domain; (b) hybridizing the analyte to the capture domain; (c) identifying the region of interest in the biological sample; (d) isolating the region of interest from the biological sample (e.g., using laser light or a scalpel to carve out the region of interest); and (e) determining (i) all or a portion of a sequence of the spatial barcode, or a complement thereof, and (ii) all or a portion of a sequence of the analyte, or a complement thereof, and using the sequences of (i) and (ii) to identify the analyte in the region of interest of the biological sample. In some embodiments, rather than carve out the region of interest laser ablation can be used to remove all biological sample not associated with the one or more regions of interest. In some embodiments, laser light and/or laser ablation degrades the hydrogel but does not damage the capture probes (e.g., capture probes can still effectively bind and capture analytes). In some embodiments, laser light and/or laser ablation substantially degrades the hydrogel and the capture probes (e.g., capture probes can no longer effectively bind and capture analytes).

In another non-limiting example, the disclosure provides a method for identifying an analyte in a region of interest from a biological sample that includes: (a) aligning the biological sample with a substrate including a hydrogel layered over a plurality of capture probes, wherein a capture probe of the plurality of capture probes includes a spatial barcode and a capture domain; (b) identifying the region of interest of the biological sample; (c) using laser ablation on a portion of the hydrogel corresponding to the region of interest of the biological sample, thereby degrading the hydrogel and exposing the capture probe; (d) contacting the biological sample with the substrate, thereby allowing the analyte present in the region of interest to hybridize to the capture probe; and (e) determining (i) all or a portion of a sequence of the spatial barcode, or a complement thereof, and (ii) all or a portion of a sequence of the analyte, or a complement thereof, and using the sequences of (i) and (ii) to identify the location of the analyte in the biological sample.

In some embodiments, laser light and/or laser ablation degrades the hydrogel but does not damage the capture probes (e.g., capture probes can still effectively bind and capture analytes). In some embodiments, laser light and/or laser ablation degrades the hydrogel and degrades the capture probes (e.g., capture probes no longer bind to and capture analytes).

In some instances, the methods of laser capture microdissection provide resolution to 1 micrometer. That is, in some instances, the methods disclosed herein can capture an area of 1 micrometer×1 micrometer as a region of interest. It is appreciated that the user can determine the size and shape of a region of interest based on various parameters and that many discrete regions of interest (e.g., 100-10,000 regions of interest) can be present in a single biological sample (e.g., a single tissue section). It is appreciated that the methods disclosed herein can capture an area of the user's choice based on the region of interest.

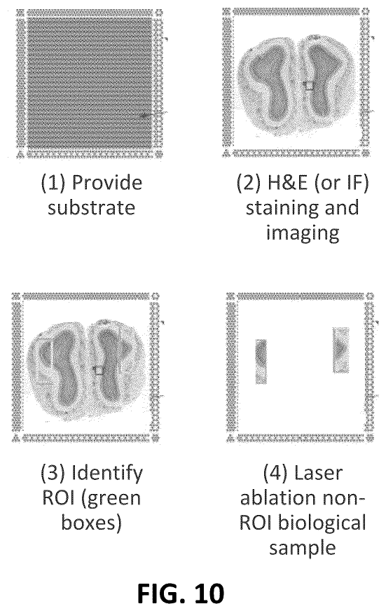

In some embodiments, laser light can be used to select a region of interest in a method for identifying an analyte in a region of interest from a biological sample. In some instances, the laser capture microdissection is used. See Emmert-Buck et al., Science (1996) 274(5289):998-1001; and Espina et al., Nature Protocols (2006) 1, 586-603; each of which is incorporated by reference in its entirety. FIG. 10 shows a non-limiting example of a method for isolating a region of interest from a biological sample using a laser light. Referring to FIG. 10, a sample is placed on a substrate (e.g., a spatial array), wherein the substrate comprises a plurality of capture probes attached to the surface of the spatial array, and wherein a capture probe of the plurality of capture probes comprises a spatial barcode and a capture domain. The sample is stained using means known in the art (e.g., H&E; IF), allowing a user to identify a region of interest (ROI) in the biological sample. As shown in FIG. 10, a laser light ablates the regions that are not associated with the ROI, thereby ablating the biological sample not associated with the region of interest. This method allows one or more analytes present in the region of interest in the biological sample to interact with the capture probe. Only a fraction of the sample (e.g., the ROI) remains after laser ablation, and this region can be subject to spatial transcriptomics, thereby determining the sequence of one or more analytes in the ROI, thereby identifying the location and abundance of the one or more analytes in the biological sample.

### C. Biological Samples and Analytes

Methods disclosed herein can be performed on any type of sample (also interchangeably called “biological sample”). In some embodiments, the sample is a fresh tissue. In some embodiments, the sample is a frozen sample. In some embodiments, the sample was previously frozen. In some embodiments, the sample is a formalin-fixed, paraffin embedded (FFPE) sample.

Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy. In some instances, the biological sample can include one or more diseased cells. A diseased cell can have altered metabolic properties, gene expression, protein expression, and/or morphologic features. Examples of diseases include inflammatory disorders, metabolic disorders, nervous system disorders, and cancer. In some instances, the biological sample includes cancer or tumor cells. Cancer cells can be derived from solid tumors, hematological malignancies, cell lines, or obtained as circulating tumor cells. In some instances, the biological sample is a heterogenous sample. In some instances, the biological sample is a heterogenous sample that includes tumor or cancer cells and/or stromal cells,

In certain embodiments, the cancer is squamous cell cancer, small-cell lung cancer, non-small cell lung cancer, gastrointestinal cancer, Hodgkin's or non-Hodgkin's lymphoma, pancreatic cancer, glioblastoma, glioma, cervical cancer, ovarian cancer, liver cancer, bladder cancer, breast cancer, colon cancer, colorectal cancer, endometrial carcinoma, myeloma, salivary gland carcinoma, kidney cancer, basal cell carcinoma, melanoma, prostate cancer, vulval cancer, thyroid cancer, testicular cancer, esophageal cancer, or a type of head or neck cancer. In certain embodiments, the cancer treated is desmoplastic melanoma, inflammatory breast cancer, thymoma, rectal cancer, anal cancer, or surgically treatable or non-surgically treatable brain stem glioma. In some embodiments, the subject is a human.

FFPE samples generally are heavily cross-linked and fragmented, and therefore this type of sample allows for limited RNA recovery using conventional detection techniques. In certain embodiments, methods of targeted RNA capture provided herein are less affected by RNA degradation associated with FFPE fixation than other methods (e.g., methods that take advantage of oligo-dT capture and reverse transcription of mRNA). In certain embodiments, methods provided herein enable sensitive measurement of specific genes of interest that otherwise might be missed with a whole transcriptomic approach.

In some instances, FFPE samples are stained (e.g., using H&E). The methods disclosed herein are compatible with H&E will allow for morphological context overlaid with transcriptomic analysis. However, depending on the need some samples may be stained with only a nuclear stain, such as staining a sample with only hematoxylin and not eosin, when location of a cell nucleus is needed.

In some embodiments, a biological sample (e.g. tissue section) can be fixed with methanol, stained with hematoxylin and eosin, and imaged. In some embodiments, fixing, staining, and imaging occurs before one or more probes are hybridized to the sample. Some embodiments of any of the workflows described herein can further include a destaining step (e.g., a hematoxylin and eosin destaining step), after imaging of the sample and prior to permeabilizing the sample. For example, destaining can be performed by performing one or more (e.g., one, two, three, four, or five) washing steps (e.g., one or more (e.g., one, two, three, four, or five) washing steps performed using a buffer including HCl). The images can be used to map spatial gene expression patterns back to the biological sample. A permeabilization enzyme can be used to permeabilize the biological sample directly on the slide.

In some embodiments, the FFPE sample is deparaffinized, permeabilized, equilibrated, and blocked before target probe oligonucleotides are added. In some embodiments, deparaffinization using xylenes. In some embodiments, deparaffinization includes multiple washes with xylenes. In some embodiments, deparaffinization includes multiple washes with xylenes followed by removal of xylenes using multiple rounds of graded alcohol followed by washing the sample with water. In some aspects, the water is deionized water. In some embodiments, equilibrating and blocking includes incubating the sample in a pre-Hyb buffer. In some embodiments, the pre-Hyb buffer includes yeast tRNA. In some embodiments, permeabilizing a sample includes washing the sample with a phosphate buffer. In some embodiments, the buffer is PBS. In some embodiments, the buffer is PBST.

The biological samples included herein comprise one or more analytes. Analytes can be broadly classified into one of two groups: nucleic acid analytes, and non-nucleic acid analytes.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte can be an organelle (e.g., nuclei or mitochondria).

Examples of nucleic acid analytes also include RNA analytes such as various types of coding and non-coding RNA. Examples of the different types of RNA analytes include messenger RNA (mRNA), ribosomal RNA (rRNA), transfer RNA (tRNA), microRNA (miRNA), and viral RNA. The RNA can be a transcript (e.g., present in a tissue section). The RNA can be small (e.g., less than 200 nucleic acid bases in length) or large (e.g., RNA greater than 200 nucleic acid bases in length). Small RNAs mainly include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

Additional examples of analytes are disclosed in WO 2020/176788 and/or U.S. Patent Application Publication No. 2020/0277663, each of which is incorporated by reference in its entirety.

### D. Methods for Preparing a Biological Sample for Spatial Analysis

### (i) Imaging and Staining

Prior to analyte migration and capture, in some instances, biological samples can be stained using a wide variety of stains and staining techniques. In some instances, the biological sample is a section on a slide (e.g., a 10μ m section). In some instances, the biological sample is dried after placement onto a glass slide. In some instances, the biological sample is dried at 42° C. In some instances, drying occurs for about 1 hour, about 2, hours, about 3 hours, or until the sections become transparent. In some instances, the biological sample can be dried overnight (e.g., in a desiccator at room temperature).

In some embodiments, a sample can be stained using any number of biological stains, including but not limited to, acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin. In some instances, the methods disclosed herein include imaging the biological sample. In some instances, imaging the sample occurs prior to deaminating the biological sample. In some instances, the sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation. In some instances, the stain is an H&E stain.

In some embodiments, the biological sample can be stained using a detectable label (e.g., radioisotopes, fluorophores, chemiluminescent compounds, bioluminescent compounds, and dyes) as described elsewhere herein. In some embodiments, a biological sample is stained using only one type of stain or one technique. In some embodiments, staining includes biological staining techniques such as H&E staining. In some embodiments, staining includes identifying analytes using fluorescently-conjugated antibodies. In some embodiments, a biological sample is stained using two or more different types of stains, or two or more different staining techniques. For example, a biological sample can be prepared by staining and imaging using one technique (e.g., H&E staining and brightfield imaging), followed by staining and imaging using another technique (e.g., IHC/IF staining and fluorescence microscopy) for the same biological sample.

In some embodiments, biological samples can be destained. Methods of destaining or discoloring a biological sample are known in the art, and generally depend on the nature of the stain(s) applied to the sample. For example, H&E staining can be destained by washing the sample in HCl, or any other acid (e.g., selenic acid, sulfuric acid, hydroiodic acid, benzoic acid, carbonic acid, malic acid, phosphoric acid, oxalic acid, succinic acid, salicylic acid, tartaric acid, sulfurous acid, trichloroacetic acid, hydrobromic acid, hydrochloric acid, nitric acid, orthophosphoric acid, arsenic acid, selenous acid, chromic acid, citric acid, hydrofluoric acid, nitrous acid, isocyanic acid, formic acid, hydrogen selenide, molybdic acid, lactic acid, acetic acid, carbonic acid, hydrogen sulfide, or combinations thereof). In some embodiments, destaining can include 1, 2, 3, 4, 5, or more washes in an acid (e.g., HCl). In some embodiments, destaining can include adding HCl to a downstream solution (e.g., permeabilization solution). In some embodiments, destaining can include dissolving an enzyme used in the disclosed methods (e.g., pepsin) in an acid (e.g., HCl) solution. In some embodiments, after destaining hematoxylin with an acid, other reagents can be added to the destaining solution to raise the pH for use in other applications. For example, SDS can be added to an acid destaining solution in order to raise the pH as compared to the acid destaining solution alone. As another example, in some embodiments, one or more immunofluorescence stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8):431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

In some embodiments, immunofluorescence or immunohistochemistry protocols (direct and indirect staining techniques) can be performed as a part of, or in addition to, the exemplary spatial workflows presented herein. For example, tissue sections can be fixed according to methods described herein. The biological sample can be transferred to an array (e.g., capture probe array), wherein analytes (e.g., proteins) are probed using immunofluorescence protocols. For example, the sample can be rehydrated, blocked, and permeabilized (3×SSC, 2% BSA, 0.1% Triton X, 1 U/μl RNAse inhibitor for 10 minutes at 4° C.) before being stained with fluorescent primary antibodies (1:100 in 3×SSC, 2% BSA, 0.1% Triton X, 1 U/μl RNAse inhibitor for 30 minutes at 4° C.). The biological sample can be washed, coverslipped (in glycerol+1 U/μl RNAse inhibitor), imaged (e.g., using a confocal microscope or other apparatus capable of fluorescent detection), washed, and processed according to analyte capture or spatial workflows described herein.

In some instances, a glycerol solution and a cover slip can be added to the sample. In some instances, the glycerol solution can include a counterstain (e.g., DAPI).

As used herein, an antigen retrieval buffer can improve antibody capture in IF/IHC protocols. An exemplary protocol for antigen retrieval can be preheating the antigen retrieval buffer (e.g., to 95° C.), immersing the biological sample in the heated antigen retrieval buffer for a predetermined time, and then removing the biological sample from the antigen retrieval buffer and washing the biological sample.

In some embodiments, optimizing permeabilization can be useful for identifying intracellular analytes. Permeabilization optimization can include selection of permeabilization agents, concentration of permeabilization agents, and permeabilization duration. Tissue permeabilization is discussed elsewhere herein.

In some embodiments, blocking an array and/or a biological sample in preparation of labeling the biological sample decreases nonspecific binding of the antibodies to the array and/or biological sample (decreases background). Some embodiments provide for blocking buffers/blocking solutions that can be applied before and/or during application of the label, wherein the blocking buffer can include a blocking agent, and optionally a surfactant and/or a salt solution. In some embodiments, a blocking agent can be bovine serum albumin (BSA), serum, gelatin (e.g., fish gelatin), milk (e.g., non-fat dry milk), casein, polyethylene glycol (PEG), polyvinyl alcohol (PVA), or polyvinylpyrrolidone (PVP), biotin blocking reagent, a peroxidase blocking reagent, levamisole, Carnoy's solution, glycine, lysine, sodium borohydride, pontamine sky blue, Sudan Black, trypan blue, FITC blocking agent, and/or acetic acid. The blocking buffer/blocking solution can be applied to the array and/or biological sample prior to and/or during labeling (e.g., application of fluorophore-conjugated antibodies) to the biological sample.

### (ii) Preparation of a Sample for Analyte Migration and Capture

In some instances, the biological sample is deparaffinized. Deparaffinization can be achieved using any method known in the art. For example, in some instances, the biological samples is treated with a series of washes that include xylene and various concentrations of ethanol. In some instances, methods of deparaffinization include treatment of xylene (e.g., three washes at 5 minutes each). In some instances, the methods further include treatment with ethanol (e.g., 100% ethanol, two washes 10 minutes each; 95% ethanol, two washes 10 minutes each; 70% ethanol, two washes 10 minutes each; 50% ethanol, two washes 10 minutes each). In some instances, after ethanol washes, the biological sample can be washed with deionized water (e.g., two washes for 5 minutes each). It is appreciated that one skilled in the art can adjust these methods to optimize deparaffinization.

In some instances, the biological sample is decrosslinked. In some instances, the biological sample is decrosslinked in a solution containing TE buffer (comprising Tris and EDTA). In some instances, the TE buffer is basic (e.g., at a pH of about 9). In some instances, decrosslinking occurs at about 50° C. to about 80° C. In some instances, decrosslinking occurs at about 70° C. In some instances, decrosslinking occurs for about 1 hour at 70° C. Just prior to decrosslinking, the biological sample can be treated with an acid (e.g., 0.1M HCl for about 1 minute). After the decrosslinking step, the biological sample can be washed (e.g., with 1× PBST).

In some instances, the methods of preparing a biological sample for analyte capture include permeabilizing the sample. In some instances, the biological sample is permeabilized using a phosphate buffer. In some instances, the phosphate buffer is PBS (e.g., 1×PBS). In some instances, the phosphate buffer is PBST (e.g., 1×PBST). In some instances, the permeabilization step is performed multiple times (e.g., 3 times at 5 minutes each).

In some instances, the methods of preparing a biological sample for analyte capture include steps of equilibrating and blocking the biological sample. In some instances, equilibrating is performed using a pre-hybridization (pre-Hyb) buffer. In some instances, the pre-Hyb buffer is RNase-free. In some instances, the pre-Hyb buffer contains no bovine serum albumin (BSA), solutions like Denhardt's, or other potentially nuclease-contaminated biological materials.

In some instances, the equilibrating step is performed multiple times (e.g., 2 times at 5 minutes each; 3 times at 5 minutes each). In some instances, the biological sample is blocked with a blocking buffer. In some instances, the blocking buffer includes a carrier such as tRNA, for example yeast tRNA such as from brewer's yeast (e.g., at a final concentration of 10-20 μg/mL). In some instances, blocking can be performed for 5, 10, 15, 20, 25, or 30 minutes.

Any of the foregoing steps can be optimized for performance. For example, one can vary the temperature. In some instances, the pre-hybridization methods are performed at room temperature. In some instances, the pre-hybridization methods are performed at 4° C. (in some instances, varying the timeframes provided herein).

E. Determining the Sequence of an Analyte after Hybridization to a Capture Domain

After an analyte from the sample has hybridized or otherwise been associated with a capture probe according to any of the methods described above in connection with the general spatial-based analytical methodology, the barcoded constructs that result from hybridization/association are analyzed.

In some embodiments, after contacting a biological sample with a substrate that includes capture probes, a removal step can optionally be performed to remove all or a portion of the biological sample from the substrate. In some embodiments, the removal step includes enzymatic and/or chemical degradation of cells of the biological sample. For example, the removal step can include treating the biological sample with an enzyme (e.g., a proteinase, e.g., proteinase K) to remove at least a portion of the biological sample from the substrate. In some embodiments, the removal step can include ablation of the tissue (e.g., laser ablation).

In some embodiments, a biological sample is not removed from the substrate. For example, the biological sample is not removed from the substrate prior to releasing a capture probe (e.g., a capture probe bound to an analyte) from the substrate. In some embodiments, such releasing comprises cleavage of the capture probe from the substrate (e.g., via a cleavage domain). In some embodiments, such releasing does not comprise releasing the capture probe from the substrate (e.g., a copy of the capture probe bound to an analyte can be made and the copy can be released from the substrate, e.g., via denaturation). In some embodiments, the biological sample is not removed from the substrate prior to analysis of an analyte bound to a capture probe after it is released from the substrate. In some embodiments, the biological sample remains on the substrate during removal of a capture probe from the substrate and/or analysis of an analyte bound to the capture probe after it is released from the substrate. In some embodiments, the biological sample remains on the substrate during removal (e.g., via denaturation) of a copy of the capture probe (e.g., complement). In some embodiments, analysis of an analyte bound to capture probe from the substrate can be performed without subjecting the biological sample to enzymatic and/or chemical degradation of the cells (e.g., permeabilized cells) or ablation of the tissue (e.g., laser ablation).

In some embodiments, at least a portion of the biological sample is not removed from the substrate. For example, a portion of the biological sample can remain on the substrate prior to releasing a capture probe (e.g., a capture prove bound to an analyte) from the substrate and/or analyzing an analyte bound to a capture probe released from the substrate. In some embodiments, at least a portion of the biological sample is not subjected to enzymatic and/or chemical degradation of the cells (e.g., permeabilized cells) or ablation of the tissue (e.g., laser ablation) prior to analysis of an analyte bound to a capture probe from the substrate.

In some embodiments, the method further includes subjecting a region of interest in the biological sample to spatial transcriptomic analysis. In some embodiments, one or more of the capture probes includes a capture domain. In some embodiments, one or more of the capture probes comprises a unique molecular identifier (UMI). In some embodiments, one or more of the capture probes comprises a cleavage domain. In some embodiments, the cleavage domain comprises a sequence recognized and cleaved by a uracil-DNA glycosylase, apurinic/apyrimidinic (AP) endonuclease (APE1), U uracil-specific excision reagent (USER), and/or an endonuclease VIII. In some embodiments, one or more capture probes do not comprise a cleavage domain and is not cleaved from the array.

In some embodiments, the method includes (i) extending the capture probe and (ii) generating second strand nucleic acid prior to the determining step (e.g., prior to determining the sequences of the spatial barcode and the analyte).

In some instances, extending the capture probe comprises a reverse transcription (RT) reaction. In some instances, the RT reaction uses the captured analyte as a template to extend the capture probe. In some instances, the reaction further includes adding a template switching oligonucleotide (TSO) that associated with the 3′ end of the extended capture probe. After addition of the TSO primer to the 3′ end of the extended capture probe, in some instances, a second strand primer is added. The second strand primer is used for second strand synthesis, which in some instances, is achieved using a polymerase enzyme.

After second strand synthesis, in some instances, the generated second strand comprises one or more primer sequences, a spatial barcode, a unique molecular identifier, a poly(T) sequence or complement thereof, part of the analyte or a complement thereof, the TSO sequence, and any combination thereof.

In some instances, after creating of the second strand, in some instances, the extended capture probe and the second strand can be denatured using any means known in the art (e.g., using a denaturing elution buffer).

In some embodiments, the extended capture probe or complement or amplicon thereof is released. The step of releasing the extended capture probe or complement or amplicon thereof from the surface of the substrate can be achieved in a number of ways. In some embodiments, an extended capture probe or a complement thereof is released from the array by nucleic acid cleavage and/or by denaturation (e.g., by heating to denature a double-stranded molecule).

In some embodiments, the extended capture probe or complement or amplicon thereof is released from the surface of the substrate (e.g., array) by physical means. For example, where the extended capture probe is indirectly immobilized on the array substrate, e.g., via hybridization to a surface probe, it can be sufficient to disrupt the interaction between the extended capture probe and the surface probe. Methods for disrupting the interaction between nucleic acid molecules include denaturing double stranded nucleic acid molecules are known in the art. A straightforward method for releasing the DNA molecules (i.e., of stripping the array of extended probes) is to use a solution that interferes with the hydrogen bonds of the double stranded molecules. In some embodiments, the extended capture probe is released by an applying heated solution, such as water or buffer, of at least 85° C., e.g., at least 90, 91, 92, 93, 94, 95, 96, 97, 98, or 99° C. In some embodiments, a solution including salts, surfactants, etc. that can further destabilize the interaction between the nucleic acid molecules is added to release the extended capture probe from the substrate.

In some embodiments, where the extended capture probe includes a cleavage domain, the extended capture probe is released from the surface of the substrate by cleavage. For example, the cleavage domain of the extended capture probe can be cleaved by any of the methods described herein. In some embodiments, the extended capture probe is released from the surface of the substrate, e.g., via cleavage of a cleavage domain in the extended capture probe, prior to the step of amplifying the extended capture probe.

In some embodiments, double-stranded extended capture probes/second strand molecules are treated to remove any unextended capture probes prior to amplification and/or analysis, e.g., sequence analysis. This can be achieved by a variety of methods, e.g., using an enzyme to degrade the unextended probes, such as an exonuclease enzyme, or purification columns.

In some embodiments, the extended capture probes/second strands are removed from the capture probe and/or biological sample and are amplified to yield quantities that are sufficient for analysis, e.g., via DNA sequencing. In some embodiments, the first strand of the extended capture probes (e.g., DNA and/or cDNA molecules) acts as a template for the amplification reaction (e.g., a polymerase chain reaction).

In some embodiments, the amplification reaction incorporates an affinity group onto the extended capture probe using a primer including the affinity group. In some embodiments, the primer includes an affinity group and the extended capture probes includes the affinity group. The affinity group can correspond to any of the affinity groups described previously.

In some instances, the extended capture probe can be amplified or copied, creating a plurality of cDNA molecules. In some embodiments, cDNA can be denatured from the capture probe template and transferred (e.g., to a clean tube) for amplification, and/or library construction. The spatially-barcoded cDNA can be amplified via PCR prior to library construction. The cDNA can then be enzymatically fragmented and size-selected in order to optimize for cDNA amplicon size. P5 and P7 sequences directed to capturing the amplicons on a sequencing flowcell (Illumina sequencing instruments) can be appended to the amplicons, i7, and i5 can be used as sample indexes, and TruSeq Read 2 can be added via End Repair, A-tailing, Adaptor Ligation, and PCR. The cDNA fragments can then be sequenced using paired-end sequencing using TruSeq Read 1 and TruSeq Read 2 as sequencing primer sites. The additional sequences are directed toward Illumina sequencing instruments or sequencing instruments that utilize those sequences; however a skilled artisan will understand that additional or alternative sequences used by other sequencing instruments or technologies are also equally applicable for use in the aforementioned methods.

In some embodiments, where a sample is barcoded directly via hybridization with capture probes or analyte capture agents hybridized, bound, or associated with either the cell surface, or introduced into the cell, as described above, sequencing can be performed on the intact sample.

Sequencing of polynucleotides can be performed by various systems. More generally, sequencing can be performed using nucleic acid amplification, polymerase chain reaction (PCR) (e.g., digital PCR and droplet digital PCR (ddPCR), quantitative PCR, real time PCR, multiplex PCR, PCR-based single plex methods, emulsion PCR), and/or isothermal amplification. Non-limiting examples of methods for sequencing genetic material include, but are not limited to, DNA hybridization methods (e.g., Southern blotting), restriction enzyme digestion methods, Sanger sequencing methods, next-generation sequencing methods (e.g., single-molecule real-time sequencing, sequence by synthesis, nanopore sequencing, and Polony sequencing), ligation methods, and microarray methods.

### III. Kits and Compositions

Also provided herein are kits and compositions that include one or more reagents for determining a location and/or an abundance of an analyte in a region of interest from a biological sample.

In some embodiments, the kit and composition includes a hydrogel comprising a plurality of capture probes. In some embodiments, the plurality capture probes are encapsulated within the hydrogel. In some embodiments, the plurality of capture probes are embedded within the hydrogel. In some embodiments, the plurality of capture probes are layered on the surface of the hydrogel. In some embodiments, the kit and composition includes one or more nanowells are deposited on the hydrogel. In some embodiments, the plurality of capture probes are layered in the nanowells. In some embodiments, the capture probe is anchored to the hydrogel at its 5′ end.

In some embodiments, the kit and composition includes reagents for isolating the region of interest of the biological sample. In some embodiments, the kit and composition includes reagents for degrading the hydrogel after the isolating step under conditions that prevent degradation of the analyte. In some embodiments, the kit and composition includes one or more degradation reagents. In some embodiments, the degradation reagent is DTT. In some embodiments, the hydrogel comprises a reagent to prevent the degradation of an analyte, for example, an RNase inhibitor. Any other suitable reagents can be used in the kit and composition described herein.

In some embodiments, the kit and composition includes a permeabilization reagent.

In some embodiments, labeled hybridization probes can be used for in situ sequencing of one or more biomarkers and/or candidate biomarkers. In some embodiments, primers can be used for amplification (e.g., clonal amplification) of a captured oligonucleotide analyte.

In some embodiments, a kit or composition can include enzyme systems for performing sequence determination. In some embodiments, the enzyme systems can include a polymerase, e.g., a DNA polymerase for second strand cDNA generation and amplification.

In some embodiments, a kit or composition can further include instructions for performing any of the methods or steps provided herein. In some embodiments, a kit or composition can include a substrate with one or more capture probes comprising a spatial barcode and a capture domain that captures a nucleic acid. In some instances, the methods includes means for sequencing the nucleic acid. In some instances, the kit includes reagents to detect and sequence the analyte. In some embodiments, the kit or composition further includes but is not limited to one or more antibodies (and/or antigen-binding antibody fragments), labeled hybridization probes, primers, or any combination thereof for visualizing one or more features of a tissue sample.

### EXAMPLES

### Example 1: Localized Spatial Tagging of a Biological Sample Using a Hydrogel Containing Capture Probes

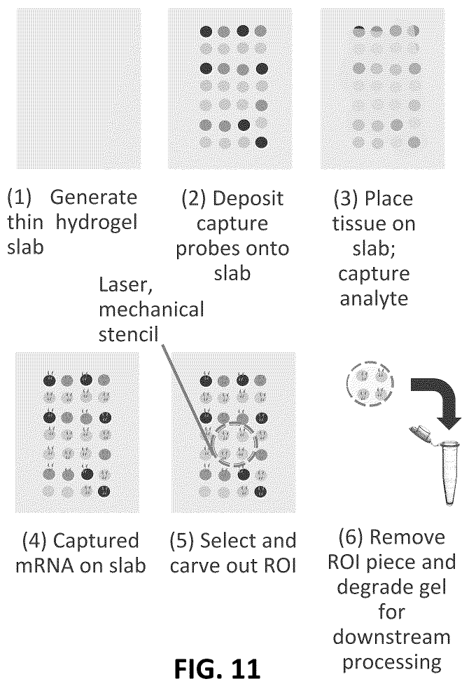

FIG. 11 shows an exemplary method for localized spatial tagging of analytes in a biological sample. In a non-limiting example, a biological sample is contacted with a hydrogel where the hydrogel includes a plurality of capture probes. A capture probe of the plurality of capture probes includes a spatial barcode and a capture domain that is capable of binding to an analyte in the biological sample.

As seen in FIG. 11, an exemplary workflow for spatially tagging analytes in a biological sample includes making a thin hydrogel slab (1) and depositing capture probes including spatial barcodes onto the thin hydrogel slab (2). The capture probes are deposited onto the hydrogel slab at known locations so that each spatial barcode is assigned to a certain position on the hydrogel slab. The biological sample (e.g., tissue section) is placed onto the hydrogel slab (3). The biological sample is permeabilized wherein analytes migrate and are captured by the capture domains of the capture probes. The sample can undergo additional processing, including capture probe extension and second strand cDNA synthesis (4). Once the processing step is complete, a region of interest of the biological sample is identified (5). The region of interest is isolated using, for example, a mechanical force to carve out and remove the region of interest from the hydrogel (5). The final step (6) includes downstream applications such as creating a sequencing ready library from the captured and processed analytes and determining the sequence of all or a portion of the sequence of the spatial barcode, or a complement thereof, and all or a portion of the sequence of the analyte, or a complement thereof from the region of interest. The determined sequence is then used to identify the analyte from the region of interest in the biological sample.

### Example 2: Localized Spatial Tagging of a Biological Sample Using a Substrate with a Hydrogel Layered Over Capture Probes

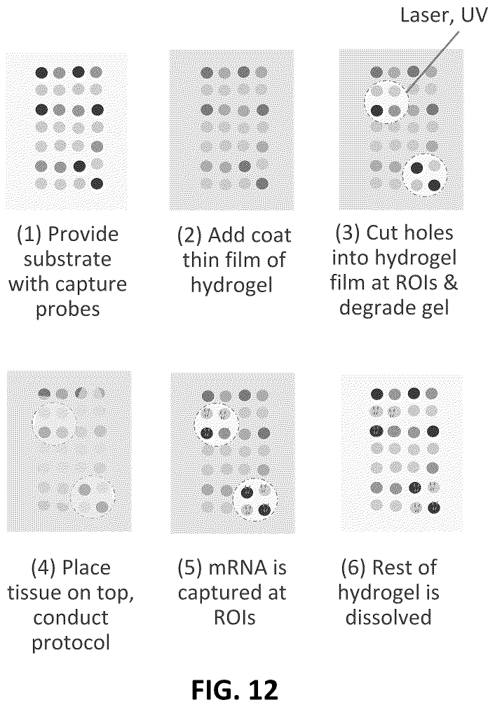

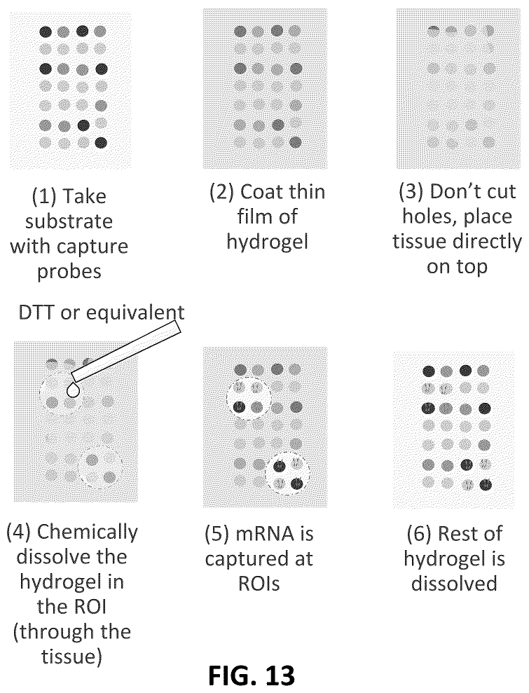

FIG. 12 and FIG. 13 show exemplary methods for localized spatial tagging of analytes in a biological sample using a substrate containing capture probes wherein the substrate is covered with a thin hydrogel layer. In a non-limiting example, a biological sample is contacted with a substrate containing capture probes affixed to the surface of the substrate and where the surface of the substrate is covered with a thin hydrogel layer. The thin hydrogel layer prevents the capture probes on the surface of the substrate from interacting with the biological sample and analytes from the biological sample. This enables a region of interest of the biological sample to be identified and selectively isolated and analyzed prior to analyte capture.

As shown in FIG. 12, an exemplary workflow for spatially tagging analytes in a biological sample includes selectively isolating a region of interest of the biological sample before the biological sample is contacted with the substrate. The substrate is printed with capture probes, wherein a capture probe comprises a spatial barcode and a capture domain (1). The substrate is covered with a hydrogel layer that covers the plurality of capture probes (2). The substrate is covered with any of the prepolymer solutions described herein. The prepolymer solution polymerizes to form a thin hydrogel layer on top of and/or around the plurality of capture probes. A region of interest of the biological sample is identified by staining the biological sample, for example with a hematoxylin and eosin (H&E) stain, by immunofluorescence, etc., to assess the pathology of the sample. An image of the stained tissue sample is generated and used to help identify one or more regions of interest. The portion(s) of the hydrogel layer on the substrate corresponding to the region(s) of interest of the biological sample is/are then degraded in order to expose the capture probes to the analytes from the defined regions of interest of the biological sample (3). Degradation is performed using, for example, laser light to degrade the portion of hydrogel associated with the region of interest. A biological sample is contacted with the substrate that includes one or more degraded hydrogel areas (4). The analytes from the biological sample at the region(s) of interest are released, for example by permeabilization, and hybridize to the capture domains on the capture probes at the region(s) of interest (5), and additional processing of the captured analytes can take place as described in Example 1. The tissue can be removed and the remainder of the hydrogel can be optionally degraded (6). After the final step (6), as in Example 1, a sequencing library is created from the processed analytes can be used for downstream analysis, including determining the sequence of all or a portion of the sequence of the spatial barcode, or a complement thereof, and all or a portion of the sequence of the analyte, or a complement thereof. The determined sequence is then used to identify the location of the analyte in the biological sample.

As shown in FIG. 13, an exemplary workflow for spatially tagging analytes in a biological sample includes selectively isolating a region of interest of the biological sample after the biological sample is contacted with the substrate. A substrate is provided that includes a plurality of capture probes, a capture probe of the plurality of capture probes includes a spatial barcode and a capture domain (1). The substrate is covered with any of the prepolymer solutions described herein. The prepolymer solution polymerizes to form a thin hydrogel layer on top of and/or around the plurality of capture probes (2). A biological sample is contacted with a substrate where the hydrogel layer prevents interaction between the plurality of capture probes and the analytes from the biological sample (3). A region of interest of the biological sample is identified by staining the biological sample, for example with a hematoxylin and eosin (H&E) stain, immunofluorescence, etc., to assess the pathology of the sample. An image of the stained sample is generated and used to help identify one or more regions of interest. In FIG. 13, the hydrogel is a DTT-sensitive hydrogel. To degrade the DTT-sensitive hydrogel, DTT is deposited onto the portion of the hydrogel layer on the substrate corresponding to the region of interest of the biological sample (4). In this case, this is done while the biological sample is contacted with the hydrogel layer. Degrading the hydrogel can also be performed concurrent with permeabilization of the tissue at the degraded locations. Following degradation of the hydrogel, the capture probe is exposed to the analytes from the biological sample. The analytes from the biological sample hybridize to the capture domains at the region(s) of interest (5) followed by additional analyte processing as previously described. The tissue can be removed and the remainder of the hydrogel can be optionally degraded (6). After the final step (6), the processed analytes can be used to create sequencing libraries for downstream analysis including determining the sequence of all or a portion of the sequence of the spatial barcode, or a complement thereof, and all or a portion of the sequence of the analyte, or a complement thereof. The determined sequence is then used to identify the location of the analyte in the biological sample.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

All publications, patent applications, patents, and other references mentioned herein are incorporated by reference in their entirety. In case of conflict, the present specification, including definitions, will control. Section headings, the materials, methods, and examples are illustrative only and not intended to be limiting.

What is claimed is:

1. A method of determining a location and/or an abundance of an analyte at a region of interest from a biological sample, the method comprising:
(a) aligning the biological sample with a substrate comprising a hydrogel layered over a plurality of capture probes, wherein a capture probe of the plurality of capture probes comprises a spatial barcode and a capture domain;
(b) identifying the region of interest in the biological sample;
(c) degrading a portion of the hydrogel corresponding to the region of interest of the biological sample, thereby exposing the capture probe;
(d) contacting the biological sample with the substrate;
(e) hybridizing the analyte at the region of interest to the capture domain; and
(f) determining (i) all or a portion of a sequence of the spatial barcode, or a complement thereof, and (ii) all or a portion of a sequence of the analyte, or a complement thereof, and using the sequences of (i) and (ii) to determine the location and/or the abundance of the analyte in the region of interest in the biological sample.

2. The method of claim 1, wherein degrading the portion of the hydrogel comprises exposing the region of interest to UV light, laser light, natural light, a heating device, a radiation device, a plasma device, a microwave device or a degradation agent, wherein the degradation agent degrades the portion of the hydrogel underneath the biological sample.

3. The method of claim 1, further comprising dissolving the hydrogel at regions outside of the region of interest.

4. The method of claim 3, further comprising, at the regions outside of the region of interest:
hybridizing an analyte outside of the region of interest to a capture domain outside of the region of interest; and
determining (i) all or a portion of a sequence of a spatial barcode of the capture domain of the region of interest, or a complement thereof, and (ii) all or a portion of a sequence of the analyte outside of the region of interest, or a complement thereof, and using the sequences of (i) and (ii) to determine the location and/or the abundance of the analyte outside of the region of interest in the biological sample.

5. The method of claim 1, wherein the hydrogel is layered over the substrate prior to contacting the biological sample with a substrate.

6. The method of claim 1, wherein degrading the portion of the hydrogel corresponding to the region of interest comprises treating the hydrogel at the region of interest with a reducing agent.

7. The method of claim 6, wherein the reducing agent comprises dithiothreitol.

8. The method of claim 6, wherein the treating the hydrogel at the region of interest with a reducing agent is performed before step (d)

9. The method of claim 1, wherein step (d) is performed before steps (b) and (c).

10. The method of claim 6, wherein the treating the hydrogel at the region of interest with a reducing agent is performed after step (d).

11. The method of claim 1, wherein the biological sample is stained using hematoxylin and eosin (H&E), immunofluorescence, or immunohistochemistry.

12. The method of claim 1, wherein the hydrogel comprises hydrogel subunits selected from the group consisting of: acrylamide, bis-acrylamide, polyacrylamide and derivatives thereof, poly(ethylene glycol) and derivatives thereof, gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, or any combination thereof.

13. The method of claim 1, wherein the hydrogel comprises one or more permeabilization reagent, wherein the permeabilization reagent comprises pepsin or proteinase K.

14. The method of claim 1, wherein the capture probe further comprises one or more functional domains, a unique molecular identifier, a cleavage domain, or any combination thereof, wherein the cleavage domain is a cleavable linker selected from the group consisting of a photocleavable linker, a UV-cleavable linker, an enzymatic cleavable linker, and a pH-sensitive cleavable linker.

15. The method of claim 1, wherein the biological sample is a fresh frozen tissue sample or formalin fixed paraffin embedded (FFPE) tissue sample.

16. The method of claim 1, wherein the analyte is an RNA molecule.

17. A kit comprising:
(a) a substrate comprising a hydrogel, wherein the substrate comprises a plurality of capture probes, wherein a capture probe of the plurality of capture probes comprises a spatial barcode and a capture domain;
(b) one or more enzymes selected from a reverse transcriptase, a DNA polymerase, or both;
(c) one or more reagents for reverse transcription or second strand synthesis
(d) a reducing agent; and
(e) instructions for performing the method of claim 1.

18. A composition comprising:
(a) a substrate comprising a hydrogel, wherein the substrate comprises a plurality of capture probes, wherein a capture probe of the plurality of capture probes comprises a spatial barcode and a capture domain;
(b) one or more enzymes selected from a reverse transcriptase, a DNA polymerase, or both;
(c) one or more reagents for reverse transcription or second strand synthesis;
(d) a reducing agent; and
(e) a biological sample.

19. A method of determining a location and/or an abundance of an analyte at a region of interest from a biological sample, the method comprising:
(a) contacting the biological sample with a substrate comprising a hydrogel, wherein the substrate comprises a plurality of capture probes, wherein a capture probe of the plurality of capture probes comprises a spatial barcode and a capture domain;
(b) hybridizing a plurality of analytes to the plurality of capture domains, wherein the plurality of analytes comprises the analyte at the region of interest;
(c) identifying the region of interest in the biological sample;
(d) isolating the region of interest of the biological sample; and
(e) determining (i) all or a portion of a sequence of the spatial barcode, or a complement thereof, and (ii) all or a portion of a sequence of the analyte, or a complement thereof, and using the sequences of (i) and (ii) to determine the location and/or the abundance of the analyte in the region of interest in the biological sample.

20. The method of claim 19, wherein isolating the region of interest of the biological sample comprises removing a portion of the hydrogel that corresponds to the region of interest using a mechanical hole punch, microsurgery, laser capture microdissection, mechanically cutting the region of interest from the biological sample, or any combination thereof
